(19) United States
(12) Patent Application Publication
(10) Pub. No.: US 2008/0293936 A1
(43) Pub. Date: Nov. 27, 2008

(54) Novel inhibitors of lysyl oxidase

(76) Inventor: Elmar Reinhold Burchardt, (US)

Correspondence Address: Elmar Burchardt, Apartment 108, Bldg. 35, 427 Village Green Drive, Ann Arbor, MI, 48105, US

(21) Appl. No.: 11/805,579

(22) Filed: May 23, 2007

(51) Int. Cl. C07D 403/04; C07D 231/08

(52) U.S. Cl. 544/238; 548/312.4

(57) ABSTRACT

The present invention concerns novel pyridazine-3-on- and pyrazol-3-on derivatives, methods of synthesizing such, a pharmaceutical agent containing pyridazine-3-on- and pyrazol-3-on derivatives as well as the use of the compounds for the prophylaxis or treatment of fibrotic diseases and/or pathologic remodelling and the use of said compounds for the expansion of stem cells.

The present invention concerns novel pyridazine-3-on- and pyrazol-3-on derivatives, methods of synthesizing such, a pharmaceutical agent containing pyridazine-3-on- and pyrazol-3-on derivatives as well as the use of the compounds for the prophylaxis or treatment of fibrotic diseases and/or pathologic remodelling and the use of the compounds for the expansion of stem cells.

In the description a number of documents are referenced to. The disclosed content of these documents, including instructions for use, is hereby incorporated by reference.

Lysyl oxidase is a key enzyme of fibrogenesis by which peptidyl residues in collagen or elastin molecules are oxidatively desalinated. Lysyl oxidase activity leads to the formation of stable covalent bonds in tropocollagen or tropoelastin that finally allows the assembly of tropocollagen into stable collagen fibers. Lysyl oxidase also occurs intracellularly and plays a role in gene regulation. For example, profibrotic genes may be upregulated in fibrotic diseases under the influence of lysyl oxidase. In aging processes an increased activity of lysyl oxidase has been measured as well, consequently an important role is attributed to it in cellular aging.

Due to its role in crosslinking collagen fibers lysyl oxidase plays a key role in the formation of pathologic collagen deposition by decreasing the degradability of collagen fibers. Inhibitors of lysyl oxidase can decrease the crosslinking of collagen and can thereby decrease the typical fibrotic remodeling of tissues. The “loose” collagen that is formed in this way can be digested by collagen degrading enzymes. By this mode of action the claimed compounds act in an antifibrotic fashion.

On the other hand, the transcription-regulating mode of action of lysyl oxidase can be downregulates by inhibition, leading to a slowing or prevention of cellular differentiation or aging processes. The claimed compounds are therefore suitable to influence cellular aging (anti-aging) and are suitable as inhibitors of differentiation of multipotent stem cells, e.g., for the repair of damaged organs or for the expansion of stem cells in vitro.

### State of the Art: Lysyl Oxidase

Lysyl oxidase is a copper-dependent amino xidase (EC 1.4.3.13), which oxidatively desaminates peptidyl lysine residues in collagen-und elastin molecules. This reaction first leads to the formation of an α-aminopeptidyl-δ-semialdehyd. Only two specific peptidyl lysine residues in the N- or C-terminal telepeptide sequence are typically oxidatively desaminated in interstitial collagens, whereas 30 to 48 peptidyl lysine residues per 1,000 peptidyl residues are modified in tropoelastin. The resulting aldehyde carbonyl groups are electrophilic and can spontaneously condensate with vicinal aminogroups of other amino groups from unmodified peptidyl lysine residues. Alternatively, adol condensation reactions with other aldehyde carbonyl groups can occur.

The reaction of lysyl oxidase thus directly leads to the formation of stable covalent links between tropocollagen or tropoelastin that eventually enable the assembly of collagen fibers from tropocollagen. The formation of covalent collagen crosslinks in collagen (I) is responsible for the characteristic 67 nm periodicity of collagen fibers seen in electron micrographs and is thus critically contributing to the formation of the stable supramolecular structure. In total four isoenzymes of lysyl oxidase have been identified so far.

Newer work has shown that lysyl oxidase also occurs intracellularly and that it plays a role in the upregulation of the expression of profibrotic genes. Thus, lysyl oxidase and collagen III often exhibit similar expression patterns in fibrotic tissues. In cell cultures it was demonstrated that lysyl oxidase activates the transcriptional activity of the human collagen (III) promoter. Further biological functions, e.g., the regulation of development, tumor suppression, cellular motility and cellular senescence are attributed to lysyl oxidase.

Lysyl oxidase plays a key role in diseases that are associated with an increased collagen deposition in the interstitial space—For example it was demonstrated in liver fibrosis that the activity of lysyl oxidase is increased several fold in patients with increased interstitial collagen deposition in comparison to normal healthy subjects. Increased concentrations of lysyl oxidase can be measured in such patients as well. Furthermore it could be demonstrated in animal models as well as in patients that the reaction products of lysyl oxidase, i.e., the dipridinium crosslinks, can be detected in strongly increased concentrations in fibrotic tissues. In addition it was shown that the degradability of deposited collagen is dependent on the degree of crosslinking. Less crosslinked collagen is degraded faster by collagenase than highly crosslinked collagen.

An increase in the activity of lysyl oxidase occurs in other fibrotic diseases—apart from liver disease—as well. For example, the intra- and extracelluar expression of lysyl oxidase is increased in skin from patients with sclerorma in comparison to normal skin. In constrictive obliterating bronchiolitis the persistence of lysyl oxidase expression may be a marker for an irreversible course of the disease. During wound healing processes an increased gene expression of lysyl oxidase was measured in the skin of rats.

In patients with premature aging (progeria) a strong increase of the expression of lysyl oxidase was measured. Furthermore it was observed in cell culture that the withdrawal of Cu²⁺ by chelators leads to an inhibition of the differentiation tendency of the cells. Cu²⁺ occurs in the active center of lysyl oxidase.

In summary, lysyl oxidase plays a key role in the formation of pathologic collagen depositions by decreasing the degradability of collagen fibers. Furthermore, lysyl oxidase potentially plays a role in the upregulation of profibrotic gene expression in fibrotic diseases. The inhibition of lysyl oxidase thus leads to an increased degradation of collagen and potientially also to a downregulation of the gene expression of profibrotic transcripts. Typical fibrotic tissue remodelling can be prevented and potentially reversed with inhibitors of lysyl oxidase

On the other hand typical aging processes can be slowed down or prevented by the inhibition of lysyl oxidase. In addition stem cells can be expanded in vitro and in vivo by inhibition of lysyl oxidase so that they are available in sufficient numbers for repair processes in tissues.

### State of the Art: Fibrotic Diseases

Fibrotic diseases include various groups of diseases which are accompanied by a qualitative change in collagen production or by an increased deposition of collagen in the extracellular space, such as systemic or localized sclerorma, liver fibroses of differing origin, such as alcoholic liver cirrhosis, biliary cirrhosis, hepatitis of viral or other genesis, idiopathic interstitial fibroses, idiopathic lung fibroses, acute pulmonary fibroses, acute respiratory distress syndrome (ARDS), perimuscular fibroses, pericentral fibroses, dermatofibromas, kidney fibroses, diabetic nephropathy, glomerulonephrites, systemic or local sclerorma, keloids, hypertrophic scar formation, joint adhesions, arthroses, myelofibroses, cicatrization of the cornea, cystic fibrosis, muscular fibroses, Duchenne's muscular dystrophy, strictures of the esophagus, Ormond's disease, Crohn's disease, ulcerative colitis and aneurysms of the large vessels.

Further fibrotic diseases can be initiated or provoked by surgical scar revisions, plastic surgery, glaucoma, cataract fibroses, cicatrizations of the cornea, graft-versus-host disease, surgical interventions performed on tendons, nerve trapping syndromes, Dupuytren's contracture, adhesions resulting from gynecological interventions, pelvic adhesions, epidural fibroses, and diseases of the thyroid gland or the parathyroid glands, and also by metastatic bone invasion, by multiple myeloma or by restenoses.

### State of the Art: Liver Fibrosis

Liver fibrosis is characterized by an increased production of extracellular matrix components which form hepatic scars. The extracellular matrix primarily consists of fibril-forming collagens, particularly collagens of type I and III, matrix-glycoconjugates such as proteoglycans, fibronectins and hyaluronic acid. The main producers of the extracellular matrix are activated hepatic stellate cells. In the healthy liver, hepatic stellate cells are quiescent cells that store retinoids and only produce small amounts of extracellular matrix proteins. By contact with fibrogenic stimuli hepatic stellate cells assume an activated phenotype which is characterized by a loss of the stored retinoid, by increased proliferation and by a morphological resemblance to myofibroblasts. In addition, activated hepatic stellate cells present with an increased expression of new genes, such as α-smooth muscle actin (α-SMA), ICAM-1, chemokines und cytokines. Fibrillary collagens of types I und III are the main expression products of activated hepatic stellate cells. In the course of collagen synthesis, C-terminal procollagen α₁(III) propeptide (PIIICP) is formed by the cleavage of procollagen α₁ (III) by procollagen C-proteinase. More recent results indicate that the cellular basis of liver fibrosis is the activation of hepatic stellate cells by fibrotic stimuli. The hepatic stellate cell changes its phenotype, proliferates und start to synthesize extracellular matrix proteins. If the rate of extracellular matrix protein synthesis exceeds the rate of collagen degradation a fibrotic scar is formed. When the fibrotic stimulus ceases to exist and the rate of collagen degradation exceeds the rate of collagen deposition, the fibrotic scar material is degraded. The resolution of fibrillary collagen—and concomitantly of the scar structure—is accompanied by an apoptosis of activated hepatic stellate cells und therefore by the disappearance of the cellular basis of fibrosis.

### State of the Art: Aging

X can denote an NR³ group, or a NR³R⁴ group or a OR⁴ group, or an SR⁴ group, wherein R³ is chosen from the group (C₁-C₆)-alkyl, trifluormethyl, (C₁-C₆)-alkanoyl, (C₁-C₆)-alkoxy, hydroxy, (C₁-C₆)-acyloxy, amino, (C₁-C₆)-acylamino, mono- and di-[(C₁-C₆)-alkylsulfonyl]amino, wherein (C₁-C₆)-alkyl and (C₁-C₆)-alkoxy can be substituted in turn with hydroxy, halogen, (C₁-C₄)-alkoxy, amino, or (C₁-C₄)-acylamino, and R⁴ is selected from (C₁-C₆)-alkandiyl, (C₁-C₆)-oxaalkandiyl, (C₁-C₆)-acylamino, (C₁-C₆)-alkylsulfonyl, wherein the (C₁-C₆)-alkandiyl group, the (C₁-C₆)-axaalkandiyl-group, the (C₁-C₆)-acylamino group, or the (C₁-C₆)-alkylsulfonyl]amino group, respectively, may in turn be substituted with hydroxy, halogen, (C₁-C₄)-alkoxy, amino, or (C₁-C₄)-acylamino.

In the present invention the residues stand for:

halogen for fluorine, chloiner, bromine or iodide,
(C₁-C₆)-alkyl for methyl, ethyl, n-propyl, i-propyl, n-butyl, i-butyl, tert.-butyl, n-pentyl, i-pentyl, tert.-pentyl, n-hexyl, i-hexyl or tert.-hexyl,
(C₂-C₆)-alkenyl for ethenyl, propenyl, butenyl, pentenyl or hexenyl,
(C₃-C₈)-cycloalkyl for cyclopropyl, cyclobutyl, cyclopentyl, cyclohexyl, cycloheptyl or cyclooctyl,
(C₁-C₆)-alkanoyl for formyl, acetyl, propanoyl, butanoyl, pentanoyl, or hexanoyl,
(C₁-C₆)-alkoxy for methoxy, ethoxy, n-proxy, i-propoxy, n-butoxy, i-butoxy, tert.-butoxy, i-pentoxy, tert.-pentoxy, n-hexoxy, i-hexoxy or tert.-hexoxy.

The compounds according to the invention can exist in stereoisomeric forms which either relate to each other as image and mirror image (enantiomers) or which do not relate to each other as image and mirror image (diastereomers). The invention also relates to the antipodes and to the racemic forms as well as to the diastereomeric mixtures. From such mixtures of enantiomers and/or diastereomers uniform stereoisomers can be isolated using methods known to experts in the field.

The invention also concerns tautomers of the compounds, depending on the structure of the particular compounds.

Within the context of the invention, preference is given to physiologically harmless salts.

In general, physiologically harmless salts are salts of the compounds according to the invention with inorganic or organic acids.

The salts and solvates of salts are preferentially selected from halogen acid salts, carbonic acid salts and sulfonic acid salts, especially preferred from hydrochloric acid, hydrobromic acid, sulfuric acid, phosphoric acid, methanesulfonic acid, ethanesulfonic acid, phenylsulfonic acid, naphthalenedisulfonic acid, toluene-sulfonic acid, acetic acid, propionic acid, lactic acid, tartaric acid, citric acid, fumaric acid, maleic acid, and benzoic acid.

In the context of the invention solvates are defined as such forms of the compounds which in solid or liquid stated form a complex by coordination with solvent molecules. Hydrates are a special form of solvates wherein the coordination occurs with water.

The 5-7-membered saturated or unsaturated heterocycle with up to 3 heteroatoms that is mentioned in the description is preferentially chosen from: furane, imidazole, isothiazole, isoxazole, pyrane, pyrazine, pyrazole, pyridazine, pyridine, pyrimidine, pyrrole, thiophen, imidazolidine, morpholine, piperidine, pyrazolidine and/or pyrrolidine. Especially preferred is a 5-7-membered saturated or unsaturated heterocycle with one N atom in the ring and, as the case may be, a further heteroatom or heterochain element chosen from N, O, SO or SO₂, or a heteroaryl with up to two further ring nitrogen atoms that is bound by a ring nitrogen atom and that can be substituted at one to three positions with identical of different halogen or (C₁-C₆)-alkyl, which in turn can be substituted with hydroxy or halogen, as the case may be.

In as far as the shorter chain residues are preferred one can refer to the respective previous definitions from which the expert can derive the shorter chain residues.

In a preferred embodiment of the present invention R¹ in formula 1 denotes a 5-7-membered saturated or unsaturated heterocycle with a further heteroatom or hetero chain that is bound to a ring nitrogen atom and that may contain one further hetero atom or hetero chain element chosen from the N, O, S, SO or S0₂, which may be substituted once or twice, with identical or different substituents that are chosen form the group halogen, (C₁-C₆)-alkyl, (C₂-C₆)-alkenyl, (C₃-C₈)-cycloalkyl, hydroxy, oxo, carboxyl, (C₁-C₆)-alkoxycarbonyl, (C₁-C₆)-alkanoyl, (C₃-C₈)-cycloalkylcarbonyl, (C₁-C₆)-alkylsulfonyl, aminocarbonyl and (C₁-C₆). alkylaminocarbonyl wherein (C₁-C₆)-alkyl and (C₁-C₆)-alkanoyl in turn can be substituted with halogen, hydroxy, (C₁-C₄)-alkoxy, (C₁-C₄)-alkoxycarbonyl, amino, mono- or di-(C₁-C₄)-alkylamino, (C₁-C₄)-alkoxycarbonylamino mono- or diarylamino, mono- or diheteroarylamino, or with a 5- or 6-membered heterocycle with up to two heteroatoms chosen from N, O and/or S.

In a further preferred embodiment R¹ denotes a 5-membered heteroaryl residue with up to two further ring nitrogen atoms that is bound to a ring nitrogen atom and which can be substituted with identical or different substituents at up to three positions with halogen, (C₁-C₆)-alkyl, which can in turn be substituted with hydroxy or halogen.

In a further preferred embodiment R¹ denotes a 5-membered heteroaryl residue with up to two further ring nitrogen atoms that is bound to a ring nitrogen atom and which can be substituted, as the case may be, with one or two substituents such as fluorine, chlorine, (C₁-C₆)-alkoxycarbonyl or (C₁-C₆)-alkyl, or (C₁-C₆)-alkylamino, which in turn may be substituted, e.g. with hydroxyl.

In a further preferred embodiment R¹ denotes an N-atom bound imidazolyl residue or a piperazinyl residue which can be substituted at the second N-atom by methyl, ethyl, 2-hydroxyethyl, 2-methoxyethyl, acetyl, tert.butoxycarbonyl or methylsulfonyl.

In a further preferred embodiment R¹ denotes the group

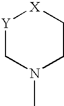

in which X and Y are identical or different and where they denote CR⁵R⁶, O, S, NR⁷ or CH₂NR⁷ respectively wherein
R⁵ and R⁶ independently of each other can denote a hydrogen, (C₁-C₄)-alkyl, which can be substituted by hydroxy, hydroxy, fluorine, carboxyl, or (C₁-C₄)-alkoxycarbonyl or they can together form a carbonyl group with the carbon atom to which they are bound, and
R⁶ denotes hydrogen, (C₂-C₄)-alkenyl, (C₃-C₆)-cycloalkyl, (C₁-C₄)-alkylsulfonyl, aminocarbonyl, (C₁-C₄). alkylaminocarbonyl, or (C₁-C₄). Alkyl which in turn can be substituted with hydroxy, methoxy, ethoxy, (C₁-C₄)-alkoxycarbonyl, amino, dimethylamino, diethylamino, pyrrolidino, piperidino or morpholino,
wherein X und Y preferentially do not simultaneously denote O, S or NR⁷.

In a preferred embodiment Y denotes a CH₂ group and X denotes NR⁷ or CH₂NR⁷.

In a preferred embodiment Ar¹ denotes phenyl which may have one or two, identical or different substituents selected from the group fluorine, chlorine, cynano, (C₁-C₆)-alkyl, trifluormethyl, formyl, acetyl, (C₁-C₆)-alkoxy, amino, (C₁-C₆)-alkylamino, hydroxy, acetoxy, pivaloyloxy, (C₁-C₆)-carboxyamino as well as formylamino, acetylamino and/or methylsulfonylamino, wherein (C₁-C₆)-alkyl and (C₁-C₆)-alkoxy may in turn be substituted with fluorine, chlorine, (C₁-C₄)-alkyl, especially methyl or ethyl, (C₁-C₄)-alkoxy, especially methoxy or ethoxy, hydroxy, amino, (C₁-C₄)-alkylamino or acetylamino.

In a preferred embodiment Ar¹ is selected from pyrrolyl, pyridyl and/or pyrimidinyl, which in turn may be substituted with fluorine, chlorine, (C₁-C₄)-alkyl, especially methyl or ethyl, (C₁-C₄)-alkoxy, especially methoxy or ethoxy, hydroxy, amino, (C₁-C₄)-alkylamino or acetylamino.

In a particularly preferred embodiment Ar¹ denotes phenyl which is in a para-position with respect to the bridging position of the phenyl ring and that has a substituent in para position chosen from OH, fluorine, chlorine, (C₁-C₄)-alkyl, hydroxy-(C₁-C₄)-alkyl or (C₁-C₄)-alkoxy-(C₁-C₄)-alkyl and, as the case may be, a second substituent in ortho position selected from hydroxy, fluorine or chlorine.

In a preferred embodiment Ar² denotes an arylthiophenyl substituent and substituted or unsubstituted biphenyl residues.

In a preferred further embodiment Ar² denotes phenyl which may have two identical or different substituents from the group, chlorine, cynano, (C₁-C₆)-alkyl, trifluormethyl, formyl, acetyl, (C₁-C₆)-alkoxy, amino, (C₁-C₆)-alkylamino, hydroxy, acetoxy, pivaloyloxy, (C₁-C₆)-carboxylamino, as formylamino, acetylamino and/or methylsulfonylamino, wherein (C₁-C₆)-alkyl and (C₁-C₆)-alkoxy in turn may be substituted with fluorine, chlorine, (C₁-C₄)-alkyl, especially methyl or ethyl, (C₁-C₄)-alkoxy, especially methoxy or ethoxy, hydroxy, amino, (C₁-C₄)-alkylamino or acetylamino.

Compounds which exhibit a particularly high medical effectiveness are depicted below:

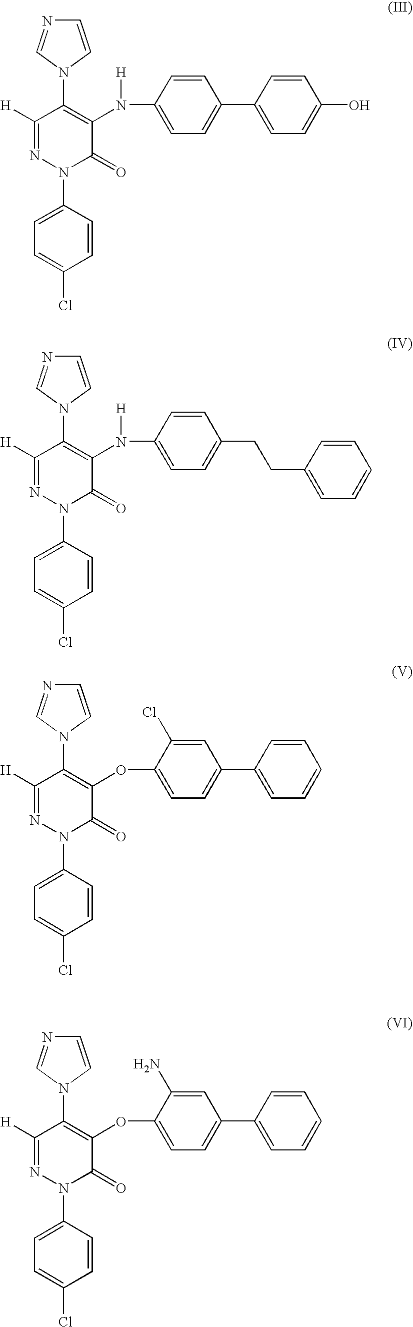

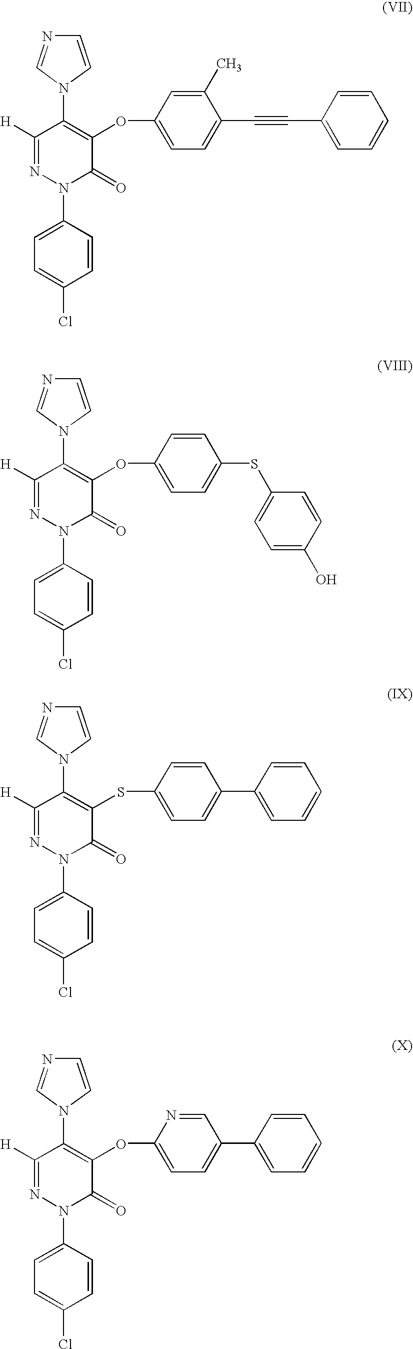

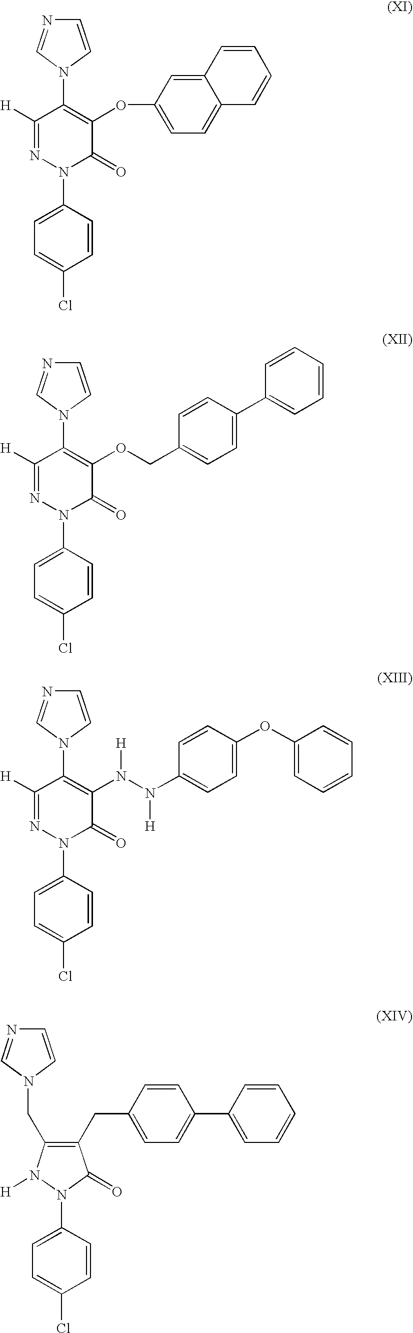

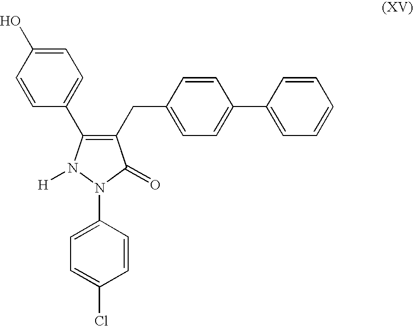

and/or und/or (depending on the compound) their tautomers, stereoisomers and salts.

The invention explicitly does not concern organic compounds of the common formula (XVI)

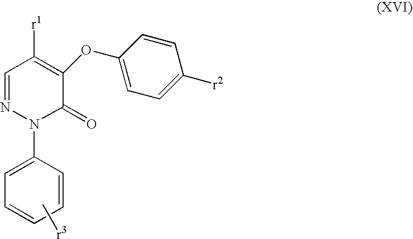

in which
r¹ denotes a 5-bis 7-membered, saturated or partially unsaturated heterocycle that is bound to a ring N-atom and that may contain a further heteroatom or heterochain chosen from N, O, S, SO or SO₂ and that may be substituted once or twice with different or identical substituents chosen from the group halogen, (C₁-C₆)-alkyl, (C₂-C₆)-alkenyl, (C₃-C₈)-cycloalkyl, hydroxy, oxo, carboxyl, (C₁-C₆)-alkoxycarbonyl, (C₁-C₆)-alkanoyl, (C₃-C₈)-cycloalkylcarbonyl, (C₁-C₆)-alkylsulfonyl, aminocarbonyl,

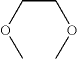

and (C₁-C₆)-alkylaminocarbonyl, wherein (C₁-C₆)-alkyl and (C₁-C₆)-alkanoyl in turn may be substituted by halogen, hydroxy, (C₁-C₄)-alkoxy, (C₁-C₄)-alkoxycarbonyl, amino, mono- or di-(C₁-C₄)-alkylamino, (C₁-C₄)-alkoxycarbonylamino or a 5- or 6-membered heterocycle with up to two heteroatoms chosen from N, O and/or S,
or
r¹ denotes a 5-membered heteroaryl with up to two further ring nitrogen atoms that can be substituted one to three times, identically or differently, with halogen, (C₁-C₆)-alkoxycarbonyl or (C₁-C₆)-alkyl, which in turn may be substituted with hydroxy or halogen,
and
r² denotes (C₆-C₁₀)-aryl that may be substituted once or twice with different or identical substituents chosen from the group halogen, nitro, cyano, (C₁-C₆)-alkyl, trifluormethyl, (C₁-C₆)-alkanoyl, (C₁-C₆)-alkoxy, hydroxy, (C₁-C₆)-acyloxy, amino, (C₁-C₆)-acylamino, mono- and di-[(C₁-C₆)-alkylsulfonyl]amino, wherein (C₁-C₆)-alkyl und (C₁-C₆)-alkoxy which in turn may be substituted with hydroxy, amino, (C₁-C₄)-alkoxy or (C₁-C₄)-acylamino hydroxy or halogen,
or
r² denotes a 5- or 6-membered heteroaryl with up to two ring nitrogen atoms that can be substituted with amino, hydroxy, halogen, (C₁-C₆)-alkyl or (C₁-C₆)-alkoxy
and
r³ denotes hydrogen, halogen, (C₁-C₆)-alkyl, trifluoromethyl, nitro, cyano, carboxyl or (C₁-C₆)-alkoxycarbonyl,
and
their salts, solvents and solvates of the salts.

The claimed compounds of the common formula I show a non-predictable valuable pharmacologic profile.

Surprisingly the claimed compounds are characterized by a high potency in regard to the inhibition of lysyl oxidase.

Surprisingly the claimed compounds bind reversibly to lysyl oxidase. The complexes consisting of lysyl oxidase and the claimed compounds can in part decay with long half life times (e.g. several hours or days). Due to this surprising characteristic the effective inhibition half life time of the claimed compounds can be substantially higher than the metabolic half life time. The time span of systemic exposition, which is in many cases determined by the metabolic half life time, can be decreased, as the case may be. Typically the pharmacologic efficacy is not decreased in this process as the target enzyme lysyl oxidase has formed stable complexes with the claimed inhibitors, which persist even when the inhibitor concentration is not sufficient to form new complexes.

A further subject of the present invention is a common method to produce compound of the common formula (Ia).

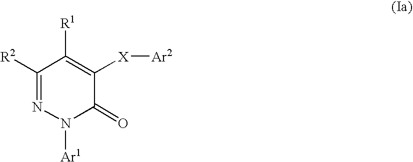

where Ar¹, Ar², R¹, R² and X are defined as above,
in which in a first reaction step a compound of the formula (XVII)

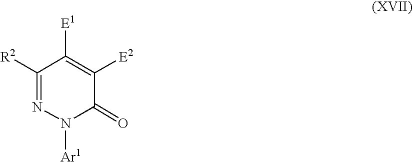

in which E¹ and E² are chosen from hydrogen, fluorine, chlorine, bromine, iodide, hydroxyl, CN, —SO₂, —SO₃, —NO₂ or PO₃, wherein chlorine bromine, iodide, CN or NO₂ are preferred,
are reacted with a compound R₁—H and a compound of formula (XVIII) is obtained,

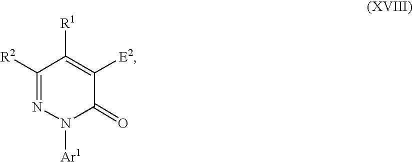

and compound (XVIII) is reacted in a second reaction step with a compound of formula (XIX),

E³-X—Ar² (XIX)

wherein E³ is chosen from H, F, Cl, Br, I or Li, Na, K, Mg_{1/2}, Zn_{1/2}, or represents another metallic center and transition metal compounds, preferably palladium and platin, can be added as catalysts.

Especially high yields can be achieved when the first reaction step is carried out in the presence of a Lewis base, such as NaI. The second reaction step is preferably carried out in the presence of an organic base, such as a nitrogen compound, e.g. a cyclic amino compound such as piperidine, piperazine, pyridine, pyrimidine, pyrrolidine, pyrazolidine, pyrrole etc.

A further embodiment of the present invention is a common method to synthesize compounds of the common formula (Ib)

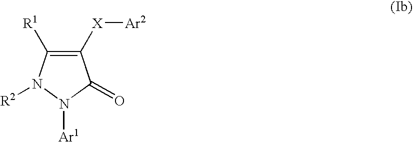

Wherein Ar¹, Ar², R¹, R² and X are defined as above.

In a first reaction step a compound of formula (XX)

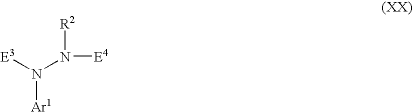

is reacted under acidic basic or neutral conditions with a compound of formula (XXI) or a derivative of (XXII) or (XXI) or a stereoisomer of (XXII),

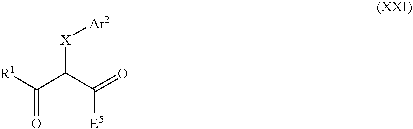

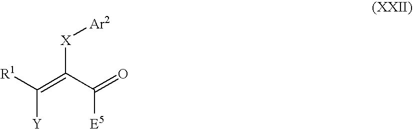

wherein Ar¹, Ar², R¹, R² and X are defined as above and E³ and E⁴ are selected from H, F, Cl, Br, I or are Li, Na, K, Mg_{1/2}, Zn_{1/2}, or another metallic center, and E⁵ is selected from F, Cl, Br, I or alkoxy or aryloxy or hetaryloxy, and Y is selected from hydroxyl, acyloxy, alkoxy, aryloxy, fluorine, chlorine, bromine, iodide and cyano, and palladium, platin or gold compounds or Lewis acids are used as additional catalysts, and thereby the hydrazine derivatives (XXIII) and (XXIV) and/or their tautomers or stereoisomers are generated, either alone or as a mixture as intermediates,

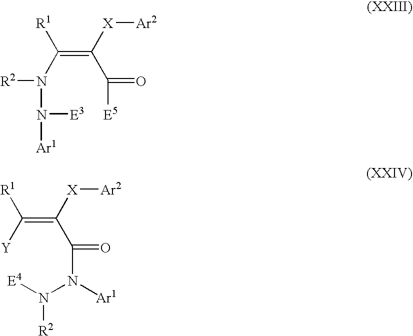

Wherein compounds (XXIII) and/or (XXIV) are incubated in a second reaction step with or without a solvent under basic, acidic or neutral conditions and are preferentially heated until cyclisation in a condensation reaction occurs and a compound of the formula (Ib) is obtained. The second reaction step may also be performed together with the first reaction step in a common incubation.

The compounds according to the invention are therefore suitable for treating liver fibroses of any genesis and fibroses which are manifested in other organs.

These fibroses include various groups of diseases which are accompanied by a qualitative change in collagen production or by an increased deposition of collagen in the extracellular space, such as liver fibroses of differing origin, such as alcoholic liver cirrhosis, biliary cirrhosis, hepatitis of viral or other genesis, idiopathic interstitial fibroses, idiopathic lung fibroses, acute pulmonary fibroses, acute respiratory distress syndrome (ARDS), perimuscular fibroses, pericentral fibroses, dermatofibromas, kidney fibroses, diabetic nephropathy, glomerulonephrites, systemic or local sclerorma, keloids, hypertrophic scar formation, joint adhesions, arthroses, myelofibrosis, cicatrization of the cornea, cystic fibrosis, muscular fibroses, Duchenne's muscular dystrophy, strictures of the esophagus, Ormond's disease, Crohn's disease, ulcerative colitis and aneurysms of the large vessels.

In addition, the invention encompasses fibrotic diseases which are initiated or provoked by surgical scar revisions, plastic surgery, glaucoma, cataract fibroses, cicatrizations of the cornea, graft-versus-host disease, surgical interventions performed on tendons, nerve trapping syndromes, Dupuytren's contracture, adhesions resulting from gynecological interventions, pelvic adhesions, epidural fibroses, and diseases of the thyroid gland or the parathyroid glands, and also by metastatic bone invasion, by multiple myeloma or by restenoses.

The biological effectiveness of the compounds can be demonstrated in vivo.

In order to demonstrate the antifibrotic effect of the substances in the liver, it is possible, for example, to use the animal model of acute or carbon tetrachloride-induced liver damage, the model of liver fibrosis due to bile duct ligature or the liver fibrosis which is induced by heterologous serum. It is also possible to use other animal models in which liver fibrosis occurs for demonstrating the antifibrotic effect.

Depending on the organ in which the fibrosis is manifested, or on the nature of the fibrotic damage, it is also possible to use animal models for other manifestations of fibrosis, for example in the heart, in the kidneys, in the lungs, in the skin or in other organs.

As a measure for the development of fibrosis it is possible to use methods that are based on the visualization of collagen on histological sections with Sirius Red/Fast Green and subsequent quantitative assessment of the fibrotic area. The reduction in collagen deposition can also be determined by hydroxyproline measurement of the fibrotic organ.

A further embodiment of the present invention concerns a drug containing one or more compounds of formula (Ia) and/or (Ib).

A further embodiment of the present invention concerns the use of the compounds of formulas (Ia) and/or (Ib) for the treatment of disease, e.g., systemic or localized sclerorma, liver fibroses of differing origin, such as alcoholic liver cirrhosis, biliary cirrhosis, hepatitis of viral or other genesis, idiopathic interstitial fibroses, idiopathic lung fibroses, acute pulmonary fibroses, acute respiratory distress syndrome (ARDS), perimuscular fibroses, pericentral fibroses, dermatofibromas, kidney fibroses, diabetic nephropathy, glomerulonephrites, systemic or local sclerorma, keloids, hypertrophic scar formation, joint adhesions, arthroses, myelofibrosis, cicatrization of the cornea, cystic fibrosis, muscular fibroses, Duchenne's muscular dystrophy, strictures of the esophagus, Ormond's disease, Crohn's disease, ulcerative colitis and aneurysms of the large vessels, and for the treatment of fibrotic diseases which are initiated or provoked by surgical scar revisions, plastic surgery, glaucoma, cataract fibroses, cicatrizations of the cornea, graft-versus-host disease, surgical interventions performed on tendons, nerve trapping syndromes, Dupuytren's contracture, adhesions resulting from gynecological interventions, pelvic adhesions, epidural fibroses, and diseases of the thyroid gland or the parathyroid glands, and also by metastatic bone invasion, by multiple myeloma or by restenoses.

A further embodiment of the present invention concerns the use of the compounds of formulas (Ia) and/or (Ib) for the treatment of liver disease.

The present invention also includes pharmaceutical preparations which, in addition to inert, nontoxic, pharmaceutically suitable adjuvants and excipients, also comprise one or more compounds of the general formula (I) and/or (Ib), or which consist of one or more active compounds of the formulae (I), and also processes for producing these preparations. These compounds can be transitioned into tablets, pills, granulates, aerosols, crémes, ointments, and emulsions by means known to the expert in the field.

In these preparations, the active compounds of the formulae (I) and/or (Ib) should be present at a concentration of from 0.05 to 99.5% by weight, preferably of from 0.5 to 95% by weight, of the total mixture.

In addition to the active compounds of the formulae (I-XV), the pharmaceutical preparations can also comprise other pharmaceutically active compounds.

The abovementioned pharmaceutical preparations can be produced in a customary manner using known methods, for example using the adjuvant(s) or excipient(s).

In general, it has proved to be advantageous, in order to achieve the desired result, to administer the active compound(s) of the formulae (I) in total quantities of from about 0.01 to about 100 mg/kg, preferably in total quantities of from about 1 mg/kg to 50 mg/kg of body weight per 24 hours, where appropriate in the form of several individual doses.

However, it can, where appropriate, be advantageous to deviate from the said quantities, depending on the nature and body weight of the individual being treated, on the individual response to the drug, on the nature and severity of the disease, on the nature of the preparation and its administration, and on the time or time interval at which the administration is effected.

### EXAMPLE 1

### Synthesis of the Compounds

The heterocyclic compound types Ia and Ib with common structural features are produced from aryl hydrazines or their salts that are suitable for storage. To produce compounds of the common structural formula (I) aryl hydrazines 1 are chemically reacted according to common procedures under neutral, basic or acid conditions with dicarbonyl compounds of type 2 (in the case of R and R²═H, it is muco choric acid) where the hydrazone 4 is formed as an intermediate which in turn cyclcondensates, preferentially in an acidic environment, to yield a heterocyclic skeleton 5. Compounds 5 with two different reactive chlorine-substituted positions can be successively reacted with different nucleophiles, preferentially under basic conditions. In this manner the substituent R¹ is introduced first and then the substituent X—Ar² wherein for both substitution steps either acidic pronucleophiles or, directly, metalloorganic compounds are used.

Synthesis steps to derive organic compounds of the common structural formula Ia:

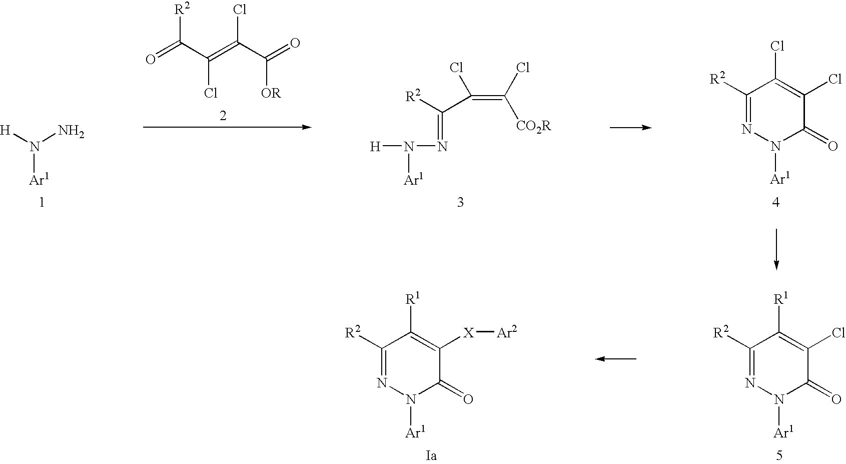

For the synthesis of compounds of the common formula II mono- (R²=H) or disubstituted hydrazines 6 are reacted according to common procedures under neutral, basic or acidic reaction conditions with dicarbonyl compounds of type 7 or of type 9. Compounds 7 which can be synthesized according to common methods are commonly preferred as reaction components since the double condensation process leads directly to the target compound Ib via the intermediate 8. With the reaction component 9 one obtains the heterocyclic skeleton 10 without Ar²—X substituents. It has to be introduced as a component using suitable electrophils as a components.

Synthesis steps to obtain compounds of the common structural formula Ib:

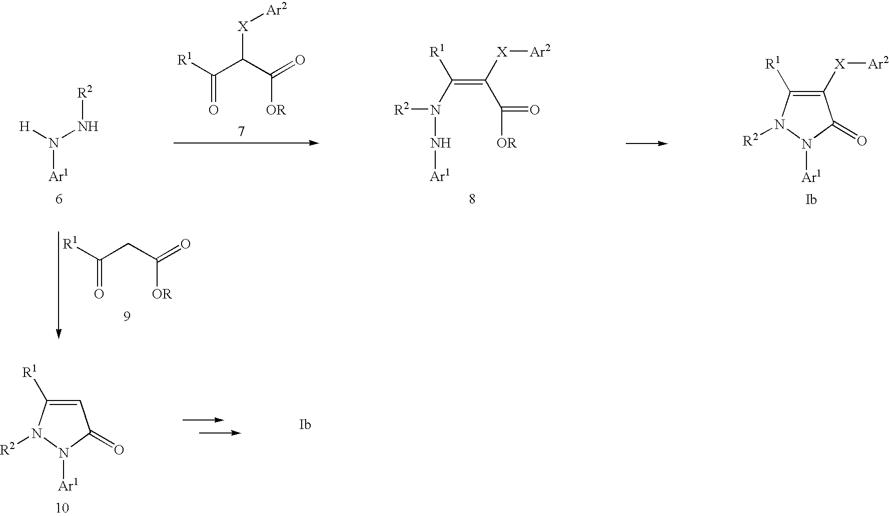

### EXAMPLE 2

### Inhibition Studies with Lysyl Oxidase

Lysyl oxidase was isolated form bovine aorta, modified according to Williams und Kagan (1985) Assessment of lysyl oxidase variants by urea gel electrophoresis: evidence against disulfide isomers as bases of the enzyme heterogeneity. Anal. Biochem. 149: 430-7, and Kagan und Sullivan (1982) Lysyl oxidase: preparation and role in elastin biosynthesis. Methods Enzymol. 82(Pt A): 637-50. The enzyme preparation was carried out at 4° C. with fresh material from bovine aorta. The aorta was homogenized for 90 seconds in 2.5. ml buffer per gram of tissue. The buffer consisted of 16 mM potassium phosphate, 150 mM sodium chloride and 1 mM phenylmethylsulfonylfluoride. The homogenized mixture was centrifuged for 20 minutes at 11,000 g. The homogenization was followed by a centrifugation step and was repeated with 150 mM sodium chloride buffer and 1 M urea buffer. The pellet that was obtained after centrifugation was homogenized in buffer plus 4 M urea, was stirred over night and centrifuged twice. The supernatants with the lysyl oxidase activity were stored each time.

To determine the specific enzyme activity of lysyl oxidase a coupled activity test with fluorescence was applied (modified according to nach Trackman P C, Zoski C G, Kagan, H M (1981) Development of a peroxidase-coupled fluorometric assay for lysyl oxidase. Anal. Biochem. 113: 336-342).

Into a 2 ml plastic cuvette the following reaction solution was pipetted:

| | | |
|---|---|---|
| | 1860 μl | 0.5 M Boraxbuffer pH = 8.2 |
| | 100 μl | 0.2 M 1,5-Diaminopentane |
| | 10 μl | 75 mg/ml Na-Homovalinate |
| | 10 μl | Horseradish peroxidase (14.5 U/μl) |
| if appropriate. | 10 μl | Inhibitor solution of suitable concentration |
| Started with: | 30 μl-100 μl | Lysyl oxidase solution |

The fluorescence of the reaction solution was determined at an excitation wavelength of 350 nm and measurement of emission was carried out at 425 nm at 37° C. Before the addition of the sample solution fluorescence was determined by calibration with 1 nmol hydrogen peroxide. This increase in fluorescence was equivalent to a turnover of 1 nmol 1,5-diaminopentane. As a positive control the time course of the increase in fluorescence when an equivalent volume of lysyl oxidase without inhibitor was added, was used. The development of fluorescence of the assay without the addition of lysyl oxidase used as a negative control. Increasing concentration of the respectively used inhibitor were used in the assay.

Using this method the concentration of the respectively used compounds at which the activity of lysyl oxidase was reduced by 50% was determined (IC₅₀). The results for some exemplary compounds are shown in table 1.

### EXAMPLE 3

### Kinetic Investigations with the Lysyl Oxidase Inhibitors

Lysyl oxidasee and lysyl oxidase inhibitors of the structural formulas (XX)-(XXIII) (Table 1) were coincubated at concentrations that were 10-fold over the respective IC₅₀ of the inhibitor and were subsequently dialyzed against 0.5 M borax buffer at pH 8.2 without added inhibitor. The dialysis buffer was exchanged twice during the first hour and subsequently three times in hourly intervals. The volume against which was dialyzed was at least 100-fold greater than the volume of the lysyl oxidase preparation in the dialysis tube. After 2, 4, 5 und 19 h lysyl oxidase activity was measured as described in Example 2. In a parallel assay a dialysis with a non-inhibited lysyl oxidase preparation was carried out to determine the effect of spontaneous activity loss during dialysis and to obtain a reference value (100% activity, positive control). From the obtained change in time-dependent activity it was possible to calculate the dissociation velocity constants of the inhibitor compounds, the half life times of the enzyme-inhibitor complexes and the association velocity constants. In the following section it is derived how these kinetic parameters can be determined form the measurement of the reaction velocity with the inhibited fraction at the timepoint t (vₜ) and the reaction velocity with the positive control (vₘₐₓ).

TABLE 1
Lysyl oxidase inhibition by test compounds

| | | | Molecular | |
|---|---|---|---|---|
| | | | weight | IC₅₀ |
| Nr. | Test comound | Structural formula | [g/mol] | [nmol/l] |
| (XX) | 2-(4-chlorophenyl)-5-(1H-imidazole-1-yl)-4-[4-(2-methoxyethyl)phenoxy]pyridazine-3(2H)-one | 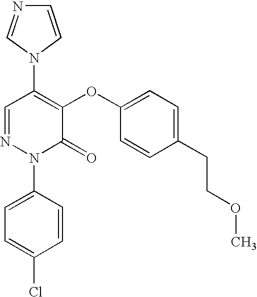 | 422.864 | 1,100 |
| (XXI) | 2-(4-chlorophenyl)-4-[4-(3-hydroxypropyl)phenoxy]-5-(1H-imidazole-1-yl)pyridazine-3(2H)-one | 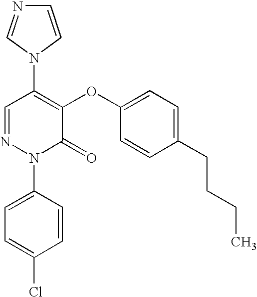 | 422.864 | 755 |
| (XXII) | 4-(1,1′-biphenyl-4-yloxy)-2-(2,4-dichlorophenyl)-5-(1H-imidazole-1-yl)pyridazine-3(2H)-one | 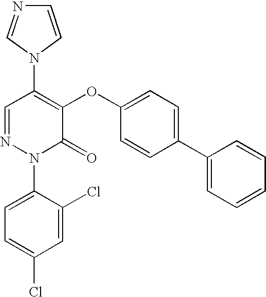 | 475.326 | 270 |
| (XXIII) | 2-(4-chlorophenyl)-5-(1H-imidazole-1-yl)-4-(4-propylphenoxy)pyridazine-3(2H)-one | 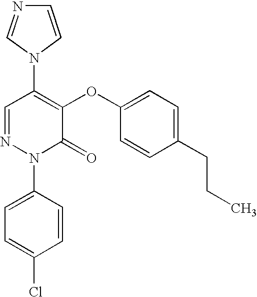 | 406.865 | 1,470 |

Derivations: During dialysis against buffer without inhibitor the complex (EI) consisting of enzyme (E) and inhibitor (I) decays with a dissociation constant of first k₋₁

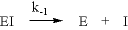

The velocity of decay is proportional to the concentration of the enzyme-inhibitor complex:

$$\frac{d [ EI ]}{d t} = {k}_{- 1} [ EI ]$$

Rearranging this equation and definite Integration from the initial enzyme-inhibitor-complex concentration ([EI]₀) to the concentration of the complex at the measurement time point t ([EI]ₜ) on the left side and from t=0 to t on the right side yields:

$${\int }_{{[ EI ]}_{0}}^{{[ EI ]}_{t}} \frac{d [ EI ]}{[ EI ]} = {k}_{- 1} {\int }_{o}^{t} d t$$

The ratio of the reaction velocity when the inhibited enzyme preparation is used at a measurement time point t (vₜ) to the reaction velocity when the positive control is used (vₘₐₓ) is dependent on the proportion of the total enzyme concentration in the assay ([E]₀) that is present as enzyme-inhibitor-complex concentration at a time point t ([EI]ₜ). The following equation describes this relationship:

$$\frac{{v}_{t}}{{v}_{max}} = \frac{{[ E ]}_{0} - {[ EI ]}_{t}}{{[ E ]}_{0}} = \frac{{[ EI ]}_{0} - {[ EI ]}_{t}}{{[ EI ]}_{0}} = 1 - \frac{{[ EI ]}_{t}}{{[ EI ]}_{0}}$$

For reasons of simplification a variable Rₜ can be defined

$${R}_{t} {=}^{def .} \frac{{[ EI ]}_{t}}{{[ EI ]}_{0}} = 1 - \frac{{v}_{t}}{{v}_{max}}$$

Solving the above differential equation and substituting Rₜ into the equation one obtains:

ln Rₜ=−k₋₁t

By linear regression with ln Rₜ and t, k₋₁ can be determined as the coefficient of slope.

Using k₋₁ one can calculate the half-life time of the enzyme-inhibitor-complexes (t_{1/2}) as:

$${t}_{1 / 2} = \frac{ln 2}{{k}_{- 1}}$$

To determine the kinetic association constant the equilibrium is considered:

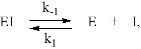

wherein the forward reaction velocitiy and the reverse reaction velocity are described by the following equations:

\vec{v}=k₋₁[EI]

[symbol]

=k₁[E][I]

During equilibrium forward and reverse reaction velocities are equal:

\vec{v}= [symbol] ,

and one obtains:

$$\frac{{k}_{- 1}}{{k}_{1}} = \frac{[ E ] [ I ]}{[ EI ]}$$

At the particular inhibitor concentration (IC₅₀) at which the reaction velocity is 50% of the reaction velocity of the positive control, the free enzyme concentration ([E]) is approximately equal to the concentration of the enzyme inhibitor complex ([EI])

[E]≈[EI]

By substitution into the equilibrium equation and by simple rearrangement one obtains a term for the association constant which is dependent on the dissociation constant determined above and on the IC₅₀:

$${k}_{1} = \frac{{k}_{- 1}}{{IC}_{50}}$$

Results Tables 2a to 2d show the results from reaction velocity measurements at the time points after 0, 120, 270 and 1140 min of dialysis against an inhibitor-free buffer. The relative reaction velocities and the derived constants are provided. In addition, the results of a regression analysis according to Example 3 are provided, from which the kinetic constants from table 3 can be calculated. Results are shown for compounds (XX)-(XIII).

TABLE 2a
Statistical analysis of (XX) (IC₅₀~270 nmol/l)

| Time point (t) [min] | vₜ/vₘₐₓ | Rₜ | In Rₜ |
|---|---|---|---|
| 0 | 0 | 1 | 0 |
| 120 | 9 × 10⁻² | 0.91 | −9.431 × 10⁻² |
| 270 | 0.28 | 0.72 | −0.3285 |
| 1,140 | 1 | 0 | n.d. |

correlation coefficient: −0.9844
ordinate intersection: 1.907×10⁻²
slope: −1.231 10^{-3 [}1/min]
k₋₁: 7.385 10^{-2 [}1/h]

TABLE 2b
Statistical analysis of (XXI) (IC₅₀~755 nmol/l)

| Time point (t) [min] | vₜ/vₘₐₓ | Rₜ | In Rₜ |
|---|---|---|---|
| 0 | 0.26 | 0.74 | −0.3011 |
| 120 | 0.27 | 0.73 | −0.3147 |
| 270 | 0.46 | 0.54 | −0.6162 |
| 1,140 | 1 | 0 | n.d. |

correlation coefficient: −0.9125
ordinate intersection: −0.2545
slope: −1.202×10^{-3 [}1/min]
k₋₁: 7.209×10^{-2 [}1/h]

TABLE 2c
Statistical analysis of (XXII) (IC₅₀~1,100 nmol/l)

| Time point (t) [min] | vₜ/vₘₐₓ | Rₜ | In Rₜ |
|---|---|---|---|
| 0 | 0 | 1 | 0 |
| 120 | 0.25 | 0.75 | −0.2877 |
| 270 | 0.36 | 0.64 | −0.4463 |
| 1,140 | 0.67 | 0.33 | −1.109 |

correlation coefficient: 0.9800
ordinate intersection: −0.1199
slope: −8.907×10⁴[1/min]
k₋₁: 5.344×10⁻² [1/h]

TABLE 2c
Statistical analysis of (XXIII) (IC₅₀~1,470 nmol/l)

| Time point (t) [min] | vₜ/vₘₐₓ | Rₜ | In Rₜ |
|---|---|---|---|
| 0 | 0.16 | 0.84 | −0.1744 |
| 120 | 0.53 | 0.47 | −0.7550 |
| 270 | 0.63 | 0.37 | −0.9943 |
| 1,140 | 0.89 | 0.11 | −2.207 |

correlation coefficient: 0.9764
ordinate intersection: −0.4147
slope: −1.616×10^{-3 [}1/min]
k₋₁: 9.694×10^{-2 [}1/h]

TABLE 3
Kinetic constants of lysysl oxidase inhibitors (XX) - (XXIII)

| Nr. | IC₅₀ [nmol/l] | k₋₁ [1/h] | k₁ [l/h/mol] | t_{1/2} [h] |
|---|---|---|---|---|
| XVI | 1,100 | 5.3 × 10⁻² | 4.9 × 10⁴ | 13.0 |
| XV | 755 | 7.2 × 10⁻² | 9.5 × 10⁴ | 9.6 |
| XIV | 270 | 7.4 × 10⁻² | 2.7 × 10⁵ | 9.4 |
| XIII | 1,470 | 9.7 × 10⁻² | 6.6 × 10⁴ | 7.3 |

The constants were calculated from the respective slopes (evaluation with linear regression)

1. Organic compounds of the common formula (I) 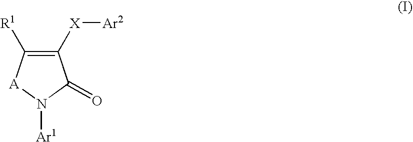
in which
R¹ denotes an alkyl residue, an aryl residue or an arylmethyl residue, which can have one or more substituents, that may be identical or different and that are chosen from the group amino, hydroxy, halogen, nitro, cyano, (C₁-C₆)-alkyl, trifluormethyl, (C₁-C₆)-alkanoyl, (C₁-C₆)-alkoxy, (C₁-C₆)-acyloxy, (C₁-C₆)-acylamino, mono- and di-[(C₁-C₆)-alkylsulfonyl]amino, wherein (C₁-C₆)-alkyl and (C₁-C₆)-alkoxy in turn can be respectively substituted with hydroxy, halogen, (C₁-C₄)-alkoxy, amino, or (C₁-C₄)-acylamino, or mono- or diarylamino, or mono- or diheteroarylamino, or R¹ can denote a substituted biphenyl residue, which can be substituted at one or several position, which can be identical or different and chosen from the group halogen, nitro, cyano, (C₁-C₆)-alkyl, trifluormethyl, (C₁-C₆)-alkanoyl, (C₁-C₆)-alkoxy, hydroxy, (C₁-C₆)-acyloxy, amino, (C₁-C₆)-acylamino, mono- and di-[(C₁-C₆)-alkylsulfonyl]amino, wherein (C₁-C₆)-alkyl und (C₁-C₆)-alkoxy in turn can be substituted by hydroxy, halogen, (C₁-C₄)-alkoxy, amino, or (C₁-C₄)-acylamino, or mono- or diarylamino, or mono- or diheteroarylamino,
or
R¹ denotes a 5- to 7-membered saturated or unsaturated heterocycle with up to three heteroatoms chosen from N, O, S, SO or SO₂, and that may be substituted once or twice as the case may be with substituents that may be identical or different and that are chosen from the group cyano, halogen, (C₁-C₆)-alkyl, (C₂-C₆)-alkenyl, (C₃-C₈)-cycloalkyl, hydroxy, oxo, carboxyl, (C₁-C₆)-alkoxycarbonyl, (C₁-C₆)-alkanoyl, (C₃-C₈)-cycloalkylcarbonyl, (C₁-C₆)-alkylsulfonyl, aminocarbonyl, 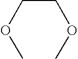
and (C₁-C₆)-alkylaminocarbonyl, and mono- or diarylamino, and mono- or diheteroarylamino, wherein (C₁-C₆)-alkyl and (C₁-C₆)-alkanoyl in turn may be substituted with halogen, hydroxy, (C₁-C₄)-alkoxy, (C₁-C₄)-alkoxycarbonyl, amino, mono- or di-(C₁-C₄)-alkylamino, (C₁-C₄)-alkoxycarbonylamino, mono- or diarylamino, mono- or diheteroarylamino or by a 5- or 6-membered heterocycle with one to two heteroatoms chosen from N, O and/or S,
and
where the structural element A denotes an imino group of the type which is integrated by a CN and an NN bond: 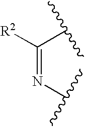
or
it denotes an amino group for the type that is integrated by two CN-bonds: 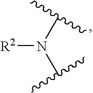
wherein
R² denotes a hydrogen, a halogen residue, an amino group, a nitro group, a cyano-group, a Hydroxy group, a (C₁-C₆)-alkyl residue, or a mono- or diarylamino group, or a mono- or diheteroarylamino group, which can have one or several subtstituents that may be identical or different and are chosen from the group halogen, nitro, cyano, (C₁-C₆)-alkyl, trifluormethyl, (C₁-C₆)-alkanoyl, (C₁-C₆)-alkoxy, hydroxy, (C₁-C₆)-acyloxy, amino, (C₁-C₆)-acylamino, mono- and di-[(C₁-C₆)-alkylsulfonyl]amino, and mono- or diarylamino, and mono- or diheteroarylamino, wherein (C₁-C₆)-alkyl and (C₁-C₆)-alkoxy may in turn be substituted hydroxy, halogen, (C₁-C₄)-alkoxy, amino, or (C₁-C₄)-acylamino,
and
Ar¹ und Ar² can be (C₅-C₁₀)-aryl residues or 5- to 7-membered saturated or unsaturated aromatic heterocycles, preferred aromatic heterocycles, that are different from each other with up to 3 hetero atoms selected from N, O, S, SO or SO₂. Furthermore the aryl residues or heterocycles denoted as Ar¹ or Ar² can for their part again be substituted with one or two aryl residues or heterocycles, for which independently the same specifications apply concerning further substituents as for the aryl residues or heterocycles directly bound to X, with the exception that they do not have to be substituted. For the aryl residues and for the biaryl residues, if applicable, and heterocycles it is furthermore required that they must contain at least one or more substituents, identical or different, that are selected from the group amino, hydroxy, halogen, nitro, cyano, (C₁-C₆)-alkyl, tri fluoro methyl, (C₁-C₆)-alkanoyl, (C₁-C₆)-alkoxy, (C₁-C₆)-acyloxy, (C₁-C₆)-alkoxycarbonyl, (C₁-C₄)-acylamino mono and di[(C₁-C₆)-alkyl sulphonyl]amino, and mono- or diarylamino, and mono- or diheteroarylamino, wherein (C₁-C₆)-alkyl and (C₁-C₆)-alkoxy for their part can be substituted with a 5- to 7-membered heteroaryl residue with up to 2 heteroatoms per ring, which can be substituted by amino, nitro, cyano, hydroxy, halogen, (C₁-C₆)-alkyl or (C₁-C₆)-alkoxy or a biphenyl residue, which can have one or several substituent which may be identical or different and that are selected from the group halogen, nitro, cyano, (C₁-C₆)-alkyl, trifluormethyl, (C₁-C₆)-alkanoyl, (C₁-C₆)-alkoxy, hydroxy, (C₁-C₆)-acyloxy, amino, (C₁-C₆)-acylamino, mono- and di-[(C₁-C₆)-alkylsulfonyl]amino, wherein (C₁-C₆)-alkyl and (C₁-C₆)-alkoxy can for their part be substituted by hydroxy, halogen, (C₁-C₄)-alkoxy, amino, or (C₁-C₄)-acylamino,
and
X can denote an O or an S-atom, an NH-group, a (C₁-C₆)-alkandiyl group, a (C₁-C₆)-oxaalkandiyl group, a (C₁-C₆)-acylamino group, a (C₁-C₆) alkylsulfonyl]amino group, wherein the (C₁-C₆)-alkandiyl group, the (C₁-C₆)-oxaalkandiyl group, the (C₁-C₆)-acylamino group or the (C₁-C₆)-alkylsulfonyl]amino group respectively can for their part once or at multiple positions be substituted by hydroxy, halogen, (C₁-C₄)-alkoxy, amino, or (C₁-C₄)-acylamino.
or
X can denote an NR³ group, or a NR³R⁴ group or a OR⁴ group, or an SR⁴ group, wherein R³ is chosen from the group (C₁-C₆)-alkyl, trifluormethyl, (C₁-C₆)-alkanoyl, (C₁-C₆)-alkoxy, hydroxy, (C₁-C₆)-acyloxy, amino, (C₁-C₆)-acylamino, mono- and di-[(C₁-C₆)-alkylsulfonyl]amino, wherein (C₁-C₆)-alkyl and (C₁-C₆)-alkoxy can be substituted in turn with hydroxy, halogen, (C₁-C₄)-alkoxy, amino, or (C₁-C₄)-acylamino,
and
R⁴ is selected from (C₁-C₆)-alkandiyl, (C₁-C₆)-oxaalkandiyl, (C₁-C₆)-acylamino, (C₁-C₆)-alkylsulfonyl, wherein the (C₁-C₆)-alkandiyl group, the (C₁-C₆)-axaalkandiyl-group, the (C₁-C₆)-acylamino group, or the (C₁-C₆)-alkylsulfonyl]amino group, respectively, may in turn be substituted with hydroxy, halogen, (C₁-C₄)-alkoxy, amino, or (C₁-C₄)-acylamino.
and/or their tautomers, stereoisomers, salts, solvates und solvates of the salts,
with the constraint that organic compounds of the common formula ((XIX) are not claimed 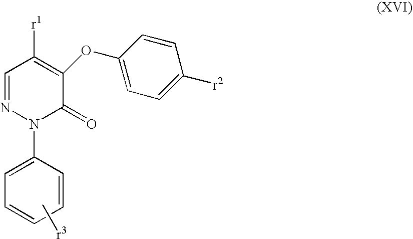
in which
r¹ denotes a 5-bis 7-membered, saturated or partially unsaturated heterocycle that is bound to a ring N-atom and that may contain a further heteroatom or heterochain chosen from N, O, S, SO or SO₂ that may be substituted once or twice with different or identical substituents chosen from the group halogen, (C₁-C₆)-alkyl, (C₂-C₆)-alkenyl, (C₃-C₈)-cycloalkyl, hydroxy, oxo, carboxyl, (C₁-C₆)-alkoxycarbonyl, (C₁-C₆)-alkanoyl, (C₃-C₈)-cycloalkylcarbonyl, (C₁-C₆)-alkylsulfonyl, aminocarbonyl, 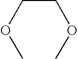
and (C₁-C₆)-alkylaminocarbonyl,
wherein (C₁-C₆)-alkyl and (C₁-C₆)-alkanoyl in turn may be substituted by halogen, hydroxy, (C₁-C₄)-alkoxy, (C₁-C₄)-alkoxycarbonyl, amino, mono- or di-(C₁-C₄)-alkylamino, (C₁-C₄)-alkoxycarbonylamino or a 5- or 6-membered heterocycle with up to two heteroatoms chosen from N, O and/or S,
or
r¹ denotes a 5-membered heteroaryl with up to two further ring nitrogen atoms that can be substituted one to three times, identically or differently, with halogen, (C₁-C₆)-alkoxycarbonyl or (C₁-C₆)-alkyl, which in turn may be substituted with hydroxy or halogen,
and
r² denotes (C₆-C₁₀)-aryl that may be substituted once or twice with different or identical substituents chosen from the group halogen, nitro, cyano, (C₁-C₆)-alkyl, trifluormethyl, (C₁-C₆)-alkanoyl, (C₁-C₆)-alkoxy, hydroxy, (C₁-C₆)-acyloxy, amino, (C₁-C₆)-acylamino, mono- and di-[(C₁-C₆)-alkylsulfonyl]amino,
wherein (C₁-C₆)-alkyl and (C₁-C₆)-alkoxy in turn may be substituted with hydroxy, amino, (C₁-C₄)-alkoxy or (C₁-C₄)-acylamino hydroxy or halogen,
or
r² denotes a 5- or 6-membered heteroaryl with up to two ring nitrogen atoms that can be substituted with amino, hydroxy, halogen, (C₁-C₆)-alkyl or (C₁-C₆)-alkoxy,
and
r³ denotes hydrogen, halogen, (C₁-C₆)-alkyl, trifluoromethyl, nitro, cyano, carboxyl or (C₁-C₆)-alkoxycarbonyl,
and their salts, solvents and solvates of the salts.

2. Organic compounds of the common formula (Ib) 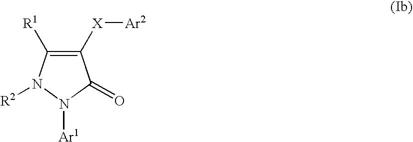
in which
R¹ denotes an alkyl residue, an aryl residue or an arylmethyl residue, which can have one or more substituents, that may be identical or different and that are chosen from the group amino, hydroxy, halogen, nitro, cyano, (C₁-C₆)-alkyl, trifluormethyl, (C₁-C₆)-alkanoyl, (C₁-C₆)-alkoxy, (C₁-C₆)-acyloxy, (C₁-C₆)-acylamino, mono- and di-[(C₁-C₆)-alkylsulfonyl]amino, wherein (C₁-C₆)-alkyl and (C₁-C₆)-alkoxy in turn can be respectively substituted with hydroxy, halogen, (C₁-C₄)-alkoxy, amino, or (C₁-C₄)-acylamino, or mono- or diarylamino, or mono- or diheteroarylamino, or R¹ can denote a substituted biphenyl residue, which can be substituted at one or several positions, which can be identical or different and chosen form the group halogen, nitro, cyano, (C₁-C₆)-alkyl, trifluormethyl, (C₁-C₆)-alkanoyl, (C₁-C₆)-alkoxy, hydroxy, (C₁-C₆)-acyloxy, amino, (C₁-C₆)-acylamino, mono- and di-[(C₁-C₆)-alkylsulfonyl]amino, wherein (C₁-C₆)-alkyl und (C₁-C₆)-alkoxy in turn can be substituted by hydroxy, halogen, (C₁-C₄)-alkoxy, amino, or (C₁-C₄)-acylamino, or mono- or diarylamino, or mono- or diheteroarylamino,
or
R¹ denotes a 5- to 7-membered saturated or unsaturated heterocycle with up to three heteroatoms chosen from N, O, S, SO or SO₂ that may be substituted once or twice as the case may be with substituents that may be identical or different and that are chosen from the group cyano, halogen, (C₁-C₆)-alkyl, (C₂-C₆)-alkenyl, (C₃-C₈)-cycloalkyl, hydroxy, oxo, carboxyl, (C₁-C₆)-alkoxycarbonyl, (C₁-C₆)-alkanoyl, (C₃-C₈)-cycloalkylcarbonyl, (C₁-C₆)-alkylsulfonyl, aminocarbonyl, 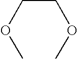
and (C₁-C₆)-alkylaminocarbonyl, and mono- or diarylamino, and mono- or diheteroarylamino, wherein (C₁-C₆)-alkyl and (C₁-C₆)-alkanoyl in turn may be substituted with halogen, hydroxy, (C₁-C₄)-alkoxy, (C₁-C₄)-alkoxycarbonyl, amino, mono- or di-(C₁-C₄)-alkylamino, (C₁-C₄)-alkoxycarbonylamino, mono- or diarylamino, mono- or diheteroarylamino or by a 5- or 6-membered heterocycle with up to two heteroatoms chosen from N, O and/or S,
and
R² denotes a hydrogen, a halogen residue, an amino group, a nitro group, a cyano-group, a hydroxy group, a (C₁-C₆)-alkyl residue, or a mono- or diarylamino group, or a mono- or diheteroarylamino group, which can have one or several subtstituents that may be identical or different and are chosen from the group halogen, nitro, cyano, (C₁-C₆)-alkyl, trifluormethyl, (C₁-C₆)-alkanoyl, (C₁-C₆)-alkoxy, hydroxy, (C₁-C₆)-acyloxy, amino, (C₁-C₆)-acylamino, mono- and di-[(C₁-C₆)-alkylsulfonyl]amino, and mono- or diarylamino, and mono- or diheteroarylamino, wherein (C₁-C₆)-alkyl and (C₁-C₆)-alkoxy may in turn be substituted by hydroxy, halogen, (C₁-C₄)-alkoxy, amino, or (C₁-C₄)-acylamino,
and
Ar¹ und Ar² can be (C₅-C₁₀)-aryl residues or 5- to 7-membered saturated or unsaturated aromatic heterocycles, preferred aromatic heterocycles, that are different from each other with up to 3 hetero atoms selected from N, O, S, SO or SO₂. Furthermore the aryl residues or heterocycles denoted as Ar¹ or Ar² can for their part again be substituted with one or two aryl residues or heterocycles, for which independently the same specifications apply concerning further substituents as for the aryl residues or heterocycles directly bound to X, with the exception that they do not have to be substituted. For the aryl residues and for the biaryl residues, if applicable, and heterocycles it is furthermore required that they must contain at least one or more substituents, identical or different, that are selected from the group amino, hydroxy, halogen, nitro, cyano, (C₁-C₆)-alkyl, tri fluoro methyl, (C₁-C₆)-alkanoyl, (C₁-C₆)-alkoxy, (C₁-C₆)-acyloxy, (C₁-C₆)-alkoxycarbonyl, (C₁-C₄)-acylamino mono and di[(C₁-C₆)-alkyl sulphonyl]amino, and mono- or diarylamino, and mono- or diheteroarylamino, wherein (C₁-C₆)-alkyl and (C₁-C₆)-alkoxy for their part can be substituted with a 5- to 7-membered heteroaryl residue with up to 2 heteroatoms per ring, which can be substituted by amino, nitro, cyano, hydroxy, halogen, (C₁-C₆)-alkyl or (C₁-C₆)-alkoxy or a biphenyl residue, which can have one or several substituents which may be identical or different and that are selected from the group halogen, nitro, cyano, (C₁-C₆)-alkyl, trifluormethyl, (C₁-C₆)-alkanoyl, (C₁-C₆)-alkoxy, hydroxy, (C₁-C₆)-acyloxy, amino, (C₁-C₆)-acylamino, mono- and di-[(C₁-C₆)-alkylsulfonyl]amino, wherein (C₁-C₆)-alkyl and (C₁-C₆)-alkoxy can for their part be substituted by hydroxy, halogen, (C₁-C₄)-alkoxy, amino, or (C₁-C₄)-acylamino,
and
X can denote an O or an S-atom, an NH-group, a (C₁-C₆)-alkandiyl group, a (C₁-C₆)-oxaalkandiyl group, a (C₁-C₆)-acylamino group, a (C₁-C₆) alkylsulfonyl]amino group, wherein the (C₁-C₆)-alkandiyl group, the (C₁-C₆)-oxaalkandiyl group, the (C₁-C₆)-acylamino group or the (C₁-C₆)-alkylsulfonyl]amino group respectively can for their part once or at multiple positions be substituted by hydroxy, halogen, (C₁-C₄)-alkoxy, amino, or (C₁-C₄)-acylamino;
or
X can denote an NR³ group, or a NR³R⁴ group or a OR⁴ group, or an SR⁴ group, wherein R³ is chosen from the group (C₁-C₆)-alkyl, trifluormethyl, (C₁-C₆)-alkanoyl, (C₁-C₆)-alkoxy, hydroxy, (C₁-C₆)-acyloxy, amino, (C₁-C₆)-acylamino, mono- and di-[(C₁-C₆)-alkylsulfonyl]amino, wherein (C₁-C₆)-alkyl and (C₁-C₆)-alkoxy can be substituted in turn with hydroxy, halogen, (C₁-C₄)-alkoxy, amino, or (C₁-C₄)-acylamino,
and
R⁴ is selected from (C₁-C₆)-alkandiyl, (C₁-C₆)-oxaalkandiyl, (C₁-C₆)-acylamino, (C₁-C₆)-alkylsulfonyl, wherein the (C₁-C₆)-alkandiyl group, the (C₁-C₆)-axaalkandiyl-group, the (C₁-C₆)-acylamino group, or the (C₁-C₆)-alkylsulfonyl]amino group, respectively, may in turn be substituted with hydroxy, halogen, (C₁-C₄)-alkoxy, amino, or (C₁-C₄)-acylamino,
and/or their tautomers, stereoisomers, salts, solvates und solvates of the salts.
